(19) United States
(12) Patent Application Publication
(10) Pub. No.: US 2009/0087831 A1
(43) Pub. Date: Apr. 2, 2009

(54) METHODS FOR EVALUATING A BACTERIOPHAGE PREPARATION

(76) Inventors: Alexander Sulakvelidze, Towson, MD (US); Shanmuga Sozhamannan, Timonium, MD (US); LeRoy Voelker, Stow, MA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/721,422

(22) PCT Filed: Dec. 17, 2004
(86) PCT No.: PCT/US03/42475

§ 371 Date: Nov. 25, 2008

Related U.S. Application Data
(60) Provisional application No. 60/529,772, filed on Dec. 17, 2003.

(51) Int. Cl. C12Q 1/70; C12N 7/01

(52) U.S. Cl. 435/5; 435/235.1

(57) ABSTRACT

A quality assurance/quality control paradigm for bacteriophage is provided.

### BACKGROUND OF THE INVENTION

1. Field of the Invention

The present invention is directed to methods for selecting, as well as monitoring and controlling the safety and efficacy of successive batches of bacteriophage prepared for industrial and/or therapeutic application.

2. Description of Related Art

Bacteriophage has been used therapeutically for much of this century. Phage were used as both prophylaxis and therapy for diseases caused by bacteria, however, the results from early studies to evaluate bacteriophage as antimicrobial agents were variable due to the uncontrolled study design and the inability to standardize reagents. Later in well designed and controlled studies it was concluded that bacteriophage were not useful as antimicrobial agents (Pyle (1936), J. Bacteriol., 12:245-61; Colvin (1932), J. Infect. Dis., 51:17-29; Boyd et al. (1944), Trans R. Soc. Trop. Med. Hyg., 37:243-62).

This initial failure of phage as antibacterial agents may have been due to the failure to select for phage that demonstrated high in vitro lytic activity prior to in vivo use. For example, phage employed may have had little or no activity against the target pathogen, were used against bacteria that were resistant due to lysogenization or the phage itself might be lysogenic for the target bacterium (Barrow et al. (1997), “Bacteriophage therapy and prophylaxis: rediscovery and renewed assessment of potential” Trends in Microbiology, 5:268-71). However, with a better understanding of the phage-bacterium interaction and of bacterial virulence factors, it was possible to conduct studies which demonstrated the in vivo anti-bacterial activity of the bacteriophage (Asheshov et al. (1937), Lancet, 1:319-20; Ward (1943), J. Infect Dis., 72:172-6; Lowbury et al. (1953), J. Gen. Microbiol., 9:524-35). In the U.S. during the 1940's Eli Lilly commercially manufactured six phage products for human use including preparations targeted towards Staphylococci, Streptococci and other respiratory pathogens.

One of the problems with utilization of bacteriophage in Western medicine and in sanitation has been consistency from batch to batch of the level of antibacterial activity in the phage preparation. This problem was acknowledged in the early days of phage therapy, as discussed above. However, technological advances in the ensuing decades (while bacteriophage therapy was out of favor in the West), open up possibilities for new protocols that may be used for characterizing bacteriophage and monitoring bacteriophage preparations. Design of such protocols (and the principles on which such protocols should be based) is the focus of the present invention.

### SUMMARY OF THE INVENTION

It is an object of the invention to provide criteria and methods for identifying bacteriophage useful in lysing pathogenic bacteria for therapeutic and sanitation purposes. The criteria are grounded in regulations that govern the safety of foods, drugs, plants, and the like, that which has an impact on human life and the human condition. Some of those regulations were promulgated by government agencies, such as the Food and Drug Administration, the U.S. Department of Agriculture and the Environmental Protection Agency. Criteria are also grounded in those characteristics that typify pathogenic bacteria, for example, strain-specific markers, lytic or lysogenic ability and the like. Based on those factors, the invention provides methods and means for identifying suitable bacteriophage. The methods taught herein can be used in the preparation of monoclonal and polyclonal lytic bacteriophage preparations.

Another object of the invention is to provide methods for monitoring the identity and quality of phage preparations for industrial and therapeutic uses. Such monitoring not only validates the active agent phage present in the preparation, but also monitors levels of contaminants, such as bacteria host by-products and media components.

Those and other objects have been achieved in the development of a series of particularized tests used alone or in combination that assess key parameters of bacteriophage preparations, such as species identification and lysogenic potential.

### BRIEF DESCRIPTION OF THE DRAWINGS

FIG. 1 is an SpeI restriction digest of the phage.

FIG. 2 provides results of a BLAST search.

### DETAILED DESCRIPTION OF THE INVENTION

Characterization of a bacteriophage-containing preparation that may be used for industrial and/or therapeutic applications should be monitored for efficacy and to ensure at least:

a) the phage preparation contains only the well-defined “monoclonal” phages (so called “monophages”) that are supposed to be included in it;

b) the phage preparation is potent against its intended bacterial targets;

c) bacterial contamination of the preparation is minimal;

d) the endotoxin level is below the danger level;

e) other pathogen-specific bacterial toxins (e.g., listeriolysin O in the case of bacteriophage directed against Listeria monocytogenes) are below the danger level; and

f) the bacteriophage is devoid of so called “undesirable” genes (e.g., bacterial toxin genes and antibiotic-resistance genes), and of bacterial 16s ribosomal RNA sequences indicative of prior lysogeny.

Potency is frequently monitored by measuring plaque-forming activity or titer. Sequential dilutions of the phage preparation are mixed with pure cultures of a target bacterial strain, and the mixture plated out under conditions suitable for growth of the bacterial strain. The concentration of the bacterial cells should be sufficient to produce an even lawn of bacteria on the plate in the absence of bacteriophage. The number of discrete plaques on the plate (bacteria-free zones representing infection by progeny of a single phage) divided by the volume of diluted stock applied to the plate, times the dilution factor, is the titer of the phage preparation. Activity of a particular phage preparation against individual strains of target bacteria may be determined by substituting the various target bacterial strains in the mixture to be plated out. While mixtures of bacteriophage or mixtures of bacterial strains may be used in this procedure, pure cultures of each (bacterial monoculture and monoclonal phage) are typically used to avoid confounding the results of the activity assay. Monoclonal bacteriophage preparations of known activity may then be mixed together to produce phage mixtures for application.

The above protocol is subjective, for example, clearness of plaques on the bacterial lawn, and therefore suffers from reproducibility. A modified procedure for assessing potency is mixing a preparation containing one or more bacteriophages with a liquid culture of targeted or control bacteria. The preparation of bacteriophages may be used without dilution, or may be diluted up to about 100,000-fold, or most preferably about 10-fold, prior to addition to the culture of targeted or control bacteria. The mixture is then incubated and lysis is determined by measuring the optical density at 600 nm in comparison to control cultures. Bacteriophage preparations are considered potent when they produce a significant reduction in the optical density at 600 nm, which indicates that clearing of the culture due to bacteriophage-mediated lysis has occurred. This assay is machine readable and thus much more objective and results can be readily standardized. This method is preferred for the measurement of the potency of individual phage as well as mixtures of two or more monoclonal bacteriophages. Some additional examples of the practical application of the method include (i) screening of various bacterial strains for susceptibility to a given phage or phage cocktail, (ii) testing for phage stability and shelf-life, and (iii) facilitating the construction of phage preparations with desired target range.

As mentioned above, a discrete plaque is an area where bacteria have been lysed by progeny of a single bacteriophage. Thus, picking and propagating phage from a discrete plaque may be expected to produce a population of genetically homogeneous bacteriophage: a monoclonal preparation. Such pure cultures are more easily characterized than heterogeneous preparations. Typically both researchers and commercial producers use monoclonal preparations, although mixtures of bacteriophage may be formulated from monoclonal cultures for particular applications. However, even if phage preparations are originally monoclonal, repeated culturing may compromise the monoclonal nature of the preparation by, e.g., contamination or mutation. Therefore, bacteriophage preparations should be monitored to confirm that they are monoclonal. Monitoring a phage preparation to confirm that it is monoclonal is facilitated by exploiting modern biotechnology techniques.

Traditionally phage preparations were examined by electron microscopy to confirm that all phage particles in the preparation had the same morphology. While relevant and still widely used, for example, used herein to assess morphology of any one stock, that procedure has two drawbacks: (1) electron microscopy will view a representative fraction of the preparation, but risks missing minor contaminants which appear only in microscope fields that were not examined, and (2) morphology of different strains may be similar enough to permit erroneously characterizing as monoclonal a preparation containing multiple different strains of similar bacteriophage. Thus, it is preferable to monitor monoclonality by applying modern analytical techniques to characterization of bacteriophage preparations to supplement or replace traditional techniques of microscopic examination.

Bacteriophage are made up of genomic nucleic acid (DNA or RNA) surrounded by a protein coat. Different strains will differ at least in the composition of the nucleic acid and frequently in the protein coat as well. Modern analytical techniques for characterizing nucleic acids and polypeptides offer significant advantages for characterizing monoclonal phage preparations and detecting deviation of bacteriophage preparations from monoclonality.

Protein fingerprinting of phage may use any suitable procedure, including but not limited to, Western blots developed against polyclonal antisera elicited against either a single phage strain or a mixture of phage strains; SDS-PAGE of phage with Coomassie blue or silver detection to identify the component proteins by molecular weight; two-dimensional electrophoresis of peptide digests using a specific protease, such as typsin, chymotrypsin, etc.; one or two-dimensional isoelectric focusing of intact phage proteins or specific protease digests; etc. Silver staining and detection modalities of similar high sensitivity for detection of minor contaminant bands in any of these techniques significantly enhance the ability to detect deviations from monoclonality.

Characterization of phage DNA for genetic homogeneity may use restriction fragment length polymorphism analysis (RFLP), pulsed field gel electrophoresis (PFGE), amplification of segments of phage DNA by the polymerase chain reaction (PCR) or alternative amplification techniques (e.g., random amplified polymorphic DNA or RAPD), and sequencing of phage genomic DNA in part or in its entirety. Developments in conjunction with the Human Genome Project and related genomic analysis research provide additional techniques for detecting minor variations in nucleic acid sequence. Bacteriophage which have RNA as their genetic material may be tested using the same techniques by converting the nucleic acid to cDNA using reverse transcriptase.

These techniques provide for detection of low levels of contamination by genetically diverse strains that produce variant bands or heterogeneous sequences on one or more of these analyses, even for bacteriophage having the same or similar morphology. Thus, these techniques enhance the potential for detecting deviations from monoclonality that might confound the characterization of the phage preparations selected for particular uses. The modern analytical techniques described herein provide for quantification of deviation from monoclonality. Preferably, a phage preparation will be considered monoclonal if it deviates by no more than the commonly accepted experimental error for the analysis, thereby effectively yielding a complete match of criteria.

Propagation of bacteriophage is routinely accomplished by culturing host bacteria that are susceptible to the phage and infecting the host culture. The phage is propagated in the bacterial host, which lyses to release copious amounts of newly produced bacteriophage. While most of the host bacteria are lysed in this process, given the large number of bacteria originally present survival of a minor fraction of the original host population could mean that applying the bacteriophage preparation will concurrently inoculate with the host bacteria. Consequently a bacteriophage preparation should be monitored for bacterial contamination. Bacterial contamination may be monitored by (1) plating 1 ml aliquots of test sample on LB agar plates or plates containing other suitable bacteriologic media and incubating replicate plates at 37° C. and 30° C. for 48 h and (2) pre-incubating 1 ml aliquots of test sample at 37° C. for 24 h Ben plating the samples on LB agar and incubating the plates for 24 h at 37° C. Any bacterial growth at the indicated times denotes contamination, Alternatively, bacterial contamination may be determined by the aerobic plate count method of the FDA Bacteriological Analytical Manual Online (http://vm.cfsan.fda.gov/˜ebam/bam-toc.html), which is incorporated herein by reference.

Although low levels of contamination by genetically divergent bacteriophage may not be that serious, contamination by lysogenic phage should always be minimized. Lysogenic bacteriophage are capable of integrating into the bacterial genome and acquiring bacterial genes that may then be transduced to yet other bacteria. In the lysogenic cycle, the phage's DNA recombines with the bacterial chromosome. Once it has inserted itself, it is known as a prophage. A host cell that carries a prophage has the potential to lyse, thus it is called a lysogenic cell.

A method for determining lysogenic potential and/or the presence of undesirable bacterial genes in the phage genome is to examine the complete nucleotide sequence of each commercialized phage to exclude phages displaying prior evidence of transduction and phages carrying undesirable genes. The methodology may be summarized as follows: (1) Isolate DNA from the candidate phages; (2) obtain the complete DNA sequence; (3) annotate the sequence using standard bioinformatics techniques (e.g. BLAST searches against all GenBank sequences); exclude all phages carrying sequences encoding any portion of 16S bacterial ribosomal RNA since phages carrying 16S sequences will have acquired these sequences from prior integration into bacterial host genomes; exclude all phages carrying sequences encoding any undesirable genes including genes encoding bacterial toxins or genes associated with drug resistance identified by comparing a complete bacteriophage sequence to all sequences contained in GenBank and other databases available through the National Center for Biotechnology Information website of the National Library of Medicine using the BLASTn program (http://www.ncbi.nlm.nih.gov/BLAST/). The nucleotide sequence may be obtained in its entirety using either the dideoxynucleotide method (see Current Protocols in Molecular Biology, Ausubel et al., ed., Wiley Interscience, NY, 1989 and periodic updates thereof), or any equivalent method. Identity of sequences is determined through a statistical parameter referred to as an e-value. An e-value of 0.00 indicates a perfect match, or absolute identity between the test sequence and a sequence in the database. In practice, significant matches are considered those with e-values ≦10⁻⁵ (see Miller et al. (2003) Complete genome sequence of the broad-host-range vibriophage KVP40: comparative genomics of a T4-related bacteriophage. J. Bacteriol. 185: 5220-33.). The cut-off e-value for this analysis is most preferably ≦10⁻⁴. The use of this e-value will provide strong assurance that no undesirable genes will be missed by this analysis. Any phages containing sequences matching any undesirable gene at the e-value ≦10⁻⁴ will be excluded from commercial phage preparations. Examples of undesirable bacterial genes include those bacterial gene products listed in 40 CFR § 725.421, which is incorporated herein by reference, see also Table 1. For bacteriophages whose genome is RNA, the same methods may be applied after first utilizing reverse transcriptase to convert the RNA to cDNA.

TABLE 1

| Sequence source | Toxin name |
|---|---|
| Corynebacterium diphtheriae & | Diphtheria toxin |
| C. ulcerans | |
| Pseudomonas aeruginosa | Exotoxin A; Proteases; |
| | Leucocidin (leukocidin, |
| | cytotoxin) |
| Shigella dysenteriae | Shigella toxin (Shiga toxin, |
| | Shigella dysenteriae type toxin, |
| | Vero cell toxin) |
| Clostridium botulinum | Neurotoxins A, B, C1, D, E, F, G |
| | (Botulinum toxins, botulinal |
| | toxins); Lysin; C2 toxin |
| Clostridium tetani | Tetanus toxin (tetanospasmin); |
| | Tetanolysin |
| Proteus mirabilis | Neurotoxin |
| Staphylococcus aureus | Alpha toxin (alpha lysin); Beta- |
| | lysin (beta toxin); Gamma lysin |
| | (Gamma toxin); Enterotoxins |
| | (SEA, SEB, SEC, SED SEE); |
| | Pyrogenic exotoxins A B; Toxic |
| | shock syndrome toxins (TSST-1); |
| | Leucocidin (leukocidin, |
| | cytotoxin) |
| Yersinia pestis | Murine toxin |
| Bacillus alve | Alveolysin |
| Bacillus cereus | Cereolysin; Enterotoxin |
| | (diarrheagenic toxin, mouse lethal |
| | factor) |
| Bacillus laterosporus | Laterosporolysin |
| Bacillus thuringiensis | Thuringiolysin |
| Clostridium bifermentans | Lysin; Lecithinase |
| Clostridium caproicum | Lysin |
| Clostridium chauvoei | Delta-toxin |
| Clostridium histolyticum | Epsilon-toxin |
| Clostridium novyi | Gamma-toxin |
| Clostridium oedematiens | Delta-toxin |
| Clostridium perfringens | Theta-toxin (Perfringolysin); |
| | Alpha-toxin (phospholipase C, |
| | lecithinase); Enterotoxin; Beta- |
| | toxin; Delta-toxin; Epsilon-toxin; |
| | Kappa-toxin |
| Clostridium septicum | Delta-toxin |
| Clostridium sordellii | Lysin |
| Listeria monocytogenes | Listeriolysin (A B) |
| Streptococcus pneumoniae | Pneumolysin |
| Bacillus anthracis | Edema factor (Factors I II); |
| | Lethal factor (Factors II III) |
| Bordetella pertussis | Adenylate cyclase (Heat-labile |
| | factor); Pertussigen (pertussis |
| | toxin, islet activating frctor, |
| | histamine sensitizing factor, |
| | lymphocytosis promoting factor) |
| Clostridium difficile | Enterotoxin (toxin A); Cytotoxin |
| | (toxin B) |
| Escherichia coli & other | Heat-labile enterotoxins (LT); |
| Enterobacteriaceae spp. | Heat-stable enterotoxins (STa, |
| | ST1 subtypes ST1a, ST1b; also |
| | STb, STII); Cytotoxin (Shiga-like |
| | toxin, Vero cell toxin) |
| Legionella pneumophila | Cytolysin |
| Vibrio cholerae & Vibrio mimicus | Cholera toxin (choleragen) |
| Other Clostridium spp. | Lecithinase |
| Corynebacterium pyogenes & other | Cytolysin (phospholipase C), |
| Corynebacterium spp. | Ovis toxin (sphingomyelinase D) |
| Aeromonas hydrophila | Aerolysin (beta-lysin, cytotoxic |
| | lysin) |
| Streptococcus pyogenes | Streptolysin S (SLS); |
| | Erythrogenic toxins (scarlet fever |
| | toxins, pyrogenic exotoxins) |
| Yersinia enterocolitica | Heat-stable enterotoxins (ST) |

Another marker for assessing lysogenic potential is determining the presence or function of integrase, which enables the viral genome to integrate into the host bacterial genome. Quantitative or semi-quantitative PCR amplification of host bacterial sequences is a quick and sensitive technique for determining maximum lysogenic contamination by the lambda int gene. The int gene sequence can be found in the Bacteriophage Lambda Complete Genome, GenBank Number NC 001416, which is incorporated herein by reference. The genomic sequence is annotated to indicate the position of the int gene. Using standard techniques and primers selected from the int sequence, quantitative or semi-quantitative PCR on nucleic acid extracts of a bacteriophage preparation will provide a measure of the level of lysogenic phage present in the preparation. Alternatively, quantitative or semi-quantitative polymerase chain reaction (PCR) amplification of host bacterial sequences is a suitable technique, and a quick screening with qualitative PCR may be used to screen for specific bacterial genes such as those listed in 40 CFR § 725.421. On a more sophisticated level, fall or partial microarrays including some or all “undesirable” genes may be constructed, and the microarrays can be used to rapidly screen various phages, or complex phage preparations, or even crude samples possibly containing phages, for the presence of these “undesirable” genes.

Even if there is no contamination of the bacteriophage preparation with living bacteria, the presence of residual bacterial products may still be cause for concern. Persistent toxins produced by bacterial cells that have since been removed or destroyed may still present a safety issue. Representative toxins are provided in Table 1. Endotoxin (lipopolysaccharide produced by Gram negative bacteria) is a well know hazard, and any bacteriophage preparation with an intended use that may result in ingestion by humans should be tested for the presence of endotoxin. Well-established procedures for monitoring endotoxin in pharmaceutical products, such as the Limulus amebocyte lysate (LAL) assay can be used for this purpose.

Certain pathogens, such as Listeria monocytogenes, may contain toxins specific to that pathogen, such as listeriolysin O (LLO). Therefore, the presence of LLO should be carefully determined in all phage preparations produced using an LLO-producing L. monocytogenes strain as the host, and all preparations should be certified not to contain LLO in concentrations that are even remotely likely to cause any adverse effects in humans (e.g., the LD₅₀ of purified LLO is approximately 26.7 μg/kg body weight of mouse (see Geoffroy et al. (1987) Purification, characterization, and toxicity of the sulfhydryl-activated hemolysin listeriolysin 0 from Listeria monocytogenes. Infect Immun. 55: 1641-6.). The same strategy should be applied to any and all other phage preparations grown on a bacterial host that is capable of producing toxins. Table 1 lists bacterial toxins produced by various bacterial species, that are considered undesirable in 40 CFR § 725.421. In the context of the above example, all phage preparations produced using, for instance, Enterotoxin (toxin A)- and Cytotoxin (toxin A)-encoding Clostridium difficile as the host, should be tested for the presence (and concentrations) of Enterotoxin (toxin A) and Cytotoxin (toxin B).

It is desirable that the content of organic compounds derived from fermentation growth medium and the fermentation biomass be as low as practicable. It is preferred that the organic content be at least about ≦125 ppm, preferably less than 50 ppm, more preferably less than 10 ppm and most preferably ≦1 ppm in a bacteriophage preparations that may be used for therapy, sanitation, or other human or animal health-related applications. Organic content may be measured by any means known in the art. A preferred method is to measure the organic content as total organic carbon using an autoanalyzer such as the Phoenix 8000, which involves oxidizing the organic carbon in a sample, detecting and quantifying the oxidized product (CO₂) and presenting the result in units of mass of carbon per volume of sample. The Phoenix 8000 uses wet chemical methods for oxidation. Specifically, the process utilizes persulfate oxidation along with UV irradiation. The sample is simultaneously exposed to persulfate ions and to UV radiation, which produces highly reactive sulfate and hydroxyl free radicals. A stream of nitrogen gas then sweeps the CO₂ produced to the detector,

It is desirable to determine the content of inorganic species in bacteriophage preparations that may be used for therapy, sanitation, or other human or animal health-related applications. Preferred species include sodium, potassium, chloride, phosphate, arsenic, and lead. Each of these species may be determined by any physical or chemical assay method known in the art. A preferred method is Inductively Coupled Plasma-Optical Emission Spectrometry (ICP). According to this method, an aqueous sample is transported, via argon gas, through a glass spray chamber to a plasma torch. Inductive coupling of time varying magnetic fields with ionized argon gas forms a high temperature (7000-9000° C.) plasma. This high temperature vaporizes the solvent, destroys chemical compounds and excites the analyte atoms. The excited atoms emit light at characteristic emission lines as they return to lower energy states. The light emitted from the plasma contains lines from all of the elements present, including argon. The Echelle Grating of the spectrometer spatially disperses the emission lines. A diode array detector measures the intensity of the emission. This intensity is proportional to the concentration of the analyte in the sample. The concentrations of analytes in the sample are calculated by comparing the intensities of the sample with that of known standards. Chloride is measured by use of an ion selective electrode, as is well-known in the art.

It is typically useful to determine other physical and chemical parameters of a bacteriophage preparation for use in industrial or therapeutic applications. Total protein, total Kjeldahl nitrogen, pH, specific gravity, total dissolved solids, percent ash, and total non-volatile solids may each be measured by known methodologies.

Bacteriophage preparations that may be used for therapy, sanitation, or other health-related applications may need to achieve broader strain specificity than is commonly associated with any single bacteriophage. A preferred method of achieving broad strain specificity is to utilize a mixture of two or more bacteriophages with desirable species and strain specificities. Once bacteriophage are mixed, it is impossible to use standard plaque-based titration methods to verify that all intended bacteriophages are present in a mixture. Therefore, alternative methodology must be employed. One preferred method is to utilize nucleotide sequences unique to each phage to design oligonucleotide primers ranging in length from approximately 18 to 30 bases. For each phage, one nucleotide corresponds in sequence to the +DNA strand (the forward primer), and the other corresponds to the −DNA strand (the reverse primer). The primers define an amplicon whose length is the number of nucleotides of phage DNA spanning the distance along the phage genome from the 5′ nucleotide of the forward primer to the 5′ nucleotide of the reverse primer, including all intervening phage nucleotides, presuming the direction of counting is along the +strand moving in the 5′ to 3′ direction from the 5′ nucleotide of the forward primer. The length of each amplicon is chosen to be both unique and readily distinguishable from other phage amplicons by agarose gel electrophoresis. Therefore, amplification by PCR or a technically equivalent amplification methodology with each set of unique primers will verify the presence of each intended bacteriophage in a mixture of bacteriophages.

Protocols for verifying the safety and efficacy of bacteriophage preparations that may be used for therapy, sanitation, or other health-related applications should include one or more of the analytical procedures described herein. For example, safety monitoring protocols should at least include an endotoxin or toxin assay for host cell contamination. and an “undesirable gene absence verification” assay. Monitoring for efficacy should include at least an activity assay. A quality control protocol could include at least one measure for each of (1) lytic activity, (2) monoclonality, (3) bacterial contamination, (4) bacterial toxin contamination, (5) lysogenic phage, (6) presence of undesirable bacterial genes; (7) organic content and (8) inorganic content including sodium, potassium, chloride, phosphate, lead, and arsenic, however, and preferably, any subcombination of those assays, as well as other assays can be selected for any one protocol. Examples of various protocols are provided below to exemplify the invention, but this invention is not limited to the particular examples chosen. Other protocols that are derived by the skilled worker from the guidance provided herein are also within the contemplation of this invention.

Because many of the assays described hereinabove and contemplated in the practice of the instant invention, such as the inorganic chemical assays, toxin assays, nucleic acid assays and proteins assays, now are amenable to automation, a protocol comprising a battery of tests can be configured to interface with a data processing unit that would control, under certain circumstances, sample preparation and deposition/introduction, data acquisition and analysis for one or more of the assays. The automation could entail the simultaneous or sequential operation of plural assays.

All references cited herein are incorporated herein by reference in entirety.

The invention will now be exemplified in the following non-limiting examples.

### EXAMPLES

### Example 1

### Plaque Assay for Activity of Bacteriophage Preparations

Sequential dilutions of the bacteriophage preparation are prepared in phosphate buffered saline (PBS), chlorine-free water, or nutrient broth (i.e., a liquid nutrient medium in which bacteria susceptible to the bacteriophage will grow). Typically, the preparation will be diluted serially in steps of 1:10 for preparations with titer of 10⁷ to 10¹². A 18-24 hour culture of a bacterial strain known to be sensitive to the bacteriophage in nutrient broth suitable for growth of the bacteria (0.1 ml) and individual dilutions of the phage preparation (1.0 ml or 1.0 ml of nutrient broth for control) are mixed and then added to 4.5 ml of 0.7% molten agar in nutrient broth at 45° C. This mixture is completely poured into a Petri dish containing 25 ml of nutrient broth solidified with 2% agar. During overnight incubation under suitable conditions, the bacteria grow in the agar and form a confluent lawn in control plates. In test plates, some bacterial cells are infected with phage; these phages replicate and lyse the initially infected cells and subsequently infect and lyse neighboring bacteria. However the agar limits the physical spread of the phage throughout the plate, resulting in small visibly clear areas called plaques on the plate where bacteriophage has destroyed bacteria within the confluent lawn of bacterial growth.

The number of plaques formed from a given volume of a given dilution of bacteriophage preparation is a reflection of the titer of the bacteriophage in the dilution, and multiplying the number of plaques by the dilution factor provides the titer of preparation. Titer may be expressed as “plaque forming units (PFU) per milliliter”, since one plaque with a distinct morphology represents one phage particle that replicated in bacteria in that area of the bacterial lawn. (The purity of a subsequent bacteriophage preparation can be ensured by removing the material in that plaque with a Pasteur pipette (a “plaque pick”) and using this material as the inoculum for further growth cycles of the phage.)

### Example 2

### Determination of Potency of a Mixture of Bacteriophages

Indicator Listeria monocytogenes strains, comprising bacterial strains susceptible to lysis by one or more bacteriophages in a mixture of bacteriophages each directed against members of the same species, were plated on modified Oxford agar NON) plates to permit isolation of a single colony following overnight incubation. After overnight incubation, a single colony of each indicator strain from the MOX plate was picked, and inoculated into a test tube containing 4.5 ml of sterile liquid nutrient medium such that there was one stain per tube. A control strain of E. coli was also prepared using an LB agar plate. The tube with the indicator strain is incubated overnight at 30° C. with 150 rpm agitation until the OD₆₀₀ reached 0.1-0.2. LMP-102™, a mixture of six bacteriophages specific for Listeria monocytogenes strains, was prepared in nutrient broth.

The component monophage of LMP-102™ are List-1 (ATCC No. PTA-5372); List-2 (ATCC No. PTA-5373); List-3 (ATCC No. PTA-5374); List-4 (ATCC No. PTA-5375); List-36 (ATCC No. PTA-5376); and List-38 (ATCC No. PTA-5377). Those phage were obtained from seawater near Baltimore. The samples and viruses contained therein were obtained practicing known methods. Phage were screened for specificity and rendered monoclonal, again, practicing methods known in the art. Table 2 provides structural characteristics of the phage.

TABLE 2

| Phage | Head (nm) | Tail (nm) |
|---|---|---|
| List-1 | 60 × 50 | 245 × 8 |
| List-2 | 56 × 44 | 272 × 8 |
| List-3 | 53 × 48 | 292 × 11 |
| List-4 | 50 × 46 | 205 × 7 |
| List-36 | 72 × 68 | 167 × 18 |
| List-38 | 60 × 65 | 258 × 9 |

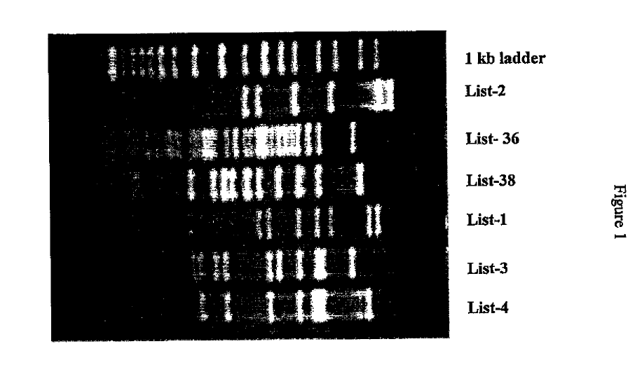

Additional diagnostic information is provided by defining PCR-amplified fragments that are specific to the six strains of phage. The primers are provided in Table 3 is SEQ ID NOS:1-12, respectively. When total DNA is digested in the SpeI, the six phage yield a diagnostic array of restriction fragments as provided in Table 4 below and in FIG. 1.

TABLE 3

| | | | Product |
|---|---|---|---|
| Phage | Primer | Sequence | size |
| List-1 | Forward | 5′-GAC AAG GCG AGC CGT GAA TGA G-3′ | 500 bp |
| | Reverse | 5′-CGA ATT GGA GGC TCA CAA ATG-3′ | |
| List-2 | Forward | 5′-CTA GTT TCA TGA AAC CTG TAG C-3′ | 1,102 bp |
| | Reverse | 5′-GCT CGG AAA CAT TGC TAA AAA T-3′ | |
| List-3 | Forward | 5′-CTC ATT TAG TAC CTG GTT AGG-3′ | 939 bp |
| | Reverse | 5′-AGA TAT GGC TAT GGA GAT GGG A-3′ | |
| List-4 | Forward | 5′-CTT TGA TAG TGA AGC CGT TGC C-3′ | 495 bp |
| | Reverse | 5′-GAA GCT TAT AGC GGC GAT ATA G-3′ | |
| List-36 | Forward | 5′-GGA CTA TCT AAA TGA TAT GGC-3′ | 1,535 bp |
| | Reverse | 5′-TTC CTT TAT CTT TTT CTT CTA G-3′ | |
| List-39 | Forward | 5′-GTG GAG AAC CCA GAG GAA AAT G-3′ | 751 bp |
| | Reverse | 5′-ATT TAA GTC GCC TAA ATC TAC-3′ | |

TABLE 4

| 1 kb | | | | | | |
|---|---|---|---|---|---|---|
| ladder | List-2 | List-36 | List-38 | List-1 | List-3 | List-4 |
| 10000 | 12000 | 7102 | 7795 | 10000 | 6955 | 8091 |
| 8000 | 10250 | 6000 | 4011 | 8042 | 6126 | 6092 |
| 6000 | 6685 | 4499 | 4067 | 5562 | 4911 | 4911 |
| 6000 | 3024 | 4067 | 3363 | 4011 | 4067 | 4657 |
| 4000 | 2804 | 3620 | 2600 | 4067 | 3355 | 4057 |
| 3500 | 2459 | 3383 | 2500 | 3251 | 3051 | 3104 |
| 3000 | | 3000 | 2162 | 2806 | 2035 | 2035 |
| 2500 | | 2806 | 2000 | 1415 | 1832 | 1367 |
| 2000 | | 2584 | 1800 | | 1555 | |
| 1500 | | 2500 | 1443 | | | |
| 1200 | | 2250 | 703 | | | |
| 1031 | | 2071 | | | | |
| 900 | | 1832 | | | | |
| 600 | | 1670 | | | | |
| 700 | | 1539 | | | | |
| 600 | | 1124 | | | | |
| 500 | | 849 | | | | |
| | | 760 | | | | |
| | | 634 | | | | |

A representative sampling of host range of the six phage is provided in Table 5 below.

TABLE 5

| Phage | Listeria monocytogenes strains lysed by phage |
|---|---|
| List-1 | Lm 4, Lm 26, Lm 54, Lm 91, Lm 107, Lm 171 |
| List-2 | Lm 30, Lm 68, Lm 69, Lm 102, Lm 122, Lm 174 |
| List-3 | Lm 38, Lm 51, Lm 54, Lm 83, Lm 101, Lm 142, Lm 107 |
| List-4 | Lm 33, Lm 38, Lm 52, Lm 78, Lm 119, Lm 120, Lm 107 |
| List-36 | Lm 27, Lm 90, Lm 101, Lm 117, Lm 140, Lm 153 |
| List-38 | Lm 37, Lm 71, Lm 04, Lm 143, Lm 148, Lm 152 |

The cost strain for each phage is uncertified.

Additional defining physical characteristics of the six phage are provided in Table 6 below.

TABLE 6

| | | | Number of open |
|---|---|---|---|
| | Size of the | | reading frames |
| Phage | penome (bp) | GC % | (ORF'S) |
| List-1 | 42.876 | 35.8 | 51 |
| List-2 | 43.030 | 35.4 | 44 |
| List-3 | 40.028 | 35.1 | 44 |
| List-4 | 40.474 | 35.1 | 45 |
| List-36 | 131.952 | 30.0 | 144 |
| List-38 | 39.339 | 30.3 | 54 |

An 0.5 ml aliquot of the bacteriophage dilution or control broth was then added to the overnight culture of the indicator bacterial strain. All tubes were then incubated overnight for 16±1 hours at 30° C. without shaking. Following incubation, the OD₆₀₀ of all tubes was measured and recorded. The bacteriophage mixture was considered potent if the OD₆₀₀ is ≦0.06. The bacteriophage mixture would not have been potent if the OD₆₀₀ were ≧0.1. Finally, the bacteriophage mixture could have had some residual potency if the OD₆₀₀ is in the range of 0.06-0.1. Results are shown in Table 7.

TABLE 7

| | | | OD₈₀₀ for Indicated Test | | | | |
|---|---|---|---|---|---|---|---|
| | | | Substance | | | | |
| LMP-102 ™ | Indicator Strains | | LMP- | LB | LMP- | LB | |
| Lot # | Species | ATCC # | 102 ™ | Broth | 102 ™ | Broth | Date Tested |
| 0103J020142 | L. monocytogenes | 15313 | 0.011 | 0.259 | 0.004 | 0.257 | Oct. 9, 2003 |
| | L. monocytogenes | 19118 | 0.005 | 0.289 | 0.002 | 0.285 | Oct. 9, 2003 |
| | L. monocytogenes | 35152 | 0.006 | 0.216 | 0.001 | 0.231 | Oct. 9, 2003 |
| | E. coli JM107 | 47014 | 0.317 | 0.434 | 0.394 | 0.442 | Oct. 9, 2003 |
| 0103J020215 | L. monocytogenes | 15313 | 0.002 | 0.280 | 0.014 | 0.257 | Oct. 9, 2003 |
| | L. monocytogenes | 19118 | 0.006 | 0.276 | 0.005 | 0.273 | Oct. 9, 2003 |
| | L. monocytogenes | 35152 | 0.002 | 0.252 | 0.005 | 0.230 | Oct. 9, 2003 |
| | E. coli JM107 | 47014 | 0.437 | 0.456 | 0.410 | 0.456 | Oct. 9, 2003 |
| 0103J020361 | L. monocytogenes | 15313 | 0.006 | 0.261 | 0.010 | 0.282 | Oct. 9, 2003 |
| | L. monocytogenes | 19118 | 0.004 | 0.269 | 0.006 | 0.275 | Oct. 9, 2003 |
| | L. monocytogenes | 35152 | 0.008 | 0.270 | 0.004 | 0.217 | Oct. 9, 2003 |
| | E. coli JM107 | 47014 | 0.419 | 0.435 | 0.415 | 0.457 | Oct. 9, 2003 |

### Example 3

### Restriction Analysis for Genotype Diversity

DNA was extracted from the indicated batches of bacteriophage specific for Listeria monocytogenes and digested with SpeI. DNA extraction was carried out by the phenol-chloroform method and restriction digestion was carried out by standard methodology (see Current Protocols in Molecular Biology, Ausubel et al., ed., Wiley-Interscience, NY, 1989 and periodic updates thereof). Digested samples were applied to a 1.2% agarose gel and electrophoresed for 50 minutes before staining with ethidium bromide. FIG. 1 shows restriction analysis of six different monophage against molecular weight standards (1 kb ladder). Each of the phage shows a different pattern. Multiple batches of a single monophage showed the same pattern for each batch.

### Example 4

### Polypeptide Analysis for Characterization of Phage Diversity

Monophage preparations were dissolved in SDS and separated on 4-12% Bis-Tris PAGE at 200V for 45 minutes by methods well-known to those skilled in the art (see Current Protocols in Molecular Biology, Ausubel, et al., ed., Wiley Interscience, NY, 1989 and periodic updates thereof). The gels were visualized by silver-staining. The analysis of the six monophage showed a different pattern.

### Example 5

### Determination of Organic and Inorganic Content of Bacteriophages

To measure total organic carbon, a Tekmar-Doorman Phoenix 8000 automated carbon analyzer or equivalent instrument was calibrated according to the manufacturer's directions with standard solutions of potassium acid phthalate at 0, 20, 50, 100, and 200 ppm carbon. Dilutions of phage samples in water were loaded onto the machine, where UV light and persulfate acted to oxidize organic compounds, permitting detection of organic carbon as CO₂, Results of the analysis of three batches of LMP-102™, a representative mixture of six bacteriophages specific for Listeria monocytogenes strains, are shown in Table 8.

TABLE 8

| | | | LMP-102 ™ | LMP-102 ™ | LMP-102 ™ | |
|---|---|---|---|---|---|---|
| | | Theoretical | Lot | Lot | Lot | LOD |
| Analysis | Method | (1X PBS) | 0103J020142 | 0103J020215 | 0103J020361 | (ppm) |
| Arsenic (ppm As) | ICP | | Not detected | Not detected | Not detected | 0.1 |
| Barium (ppm Ba) | ICP | — | Not detected | Not detected | Not detected | 0.2 |
| Cadmium (ppm Cd) | ICP | | Not detected | Not detected | Not detected | 0.03 |
| Calcium (ppm Ca) | ICP | — | Not detected | Not detected | Not detected | 0.5 |
| Chromium (ppm Cr) | ICP | — | Not detected | Not detected | Not detected | 0.15 |
| Cobalt (ppm Co) | ICP | — | <0.499 | Not detected | Not detected | 0.1 |
| Copper (ppm Cu) | ICP | — | Not detected | Not detected | Not detected | 0.1 |
| Iron (ppm Fe) | ICP | — | Not detected | Not detected | Not detected | 0.04 |
| Lead (ppm Pb) | ICP | | Not detected | Not detected | Not detected | 0.5 |
| Magnesium (ppm Mg) | ICP | — | Not detected | Not detected | Not detected | 0.8 |
| Manganese (ppm Mn) | ICP | — | Not detected | Not detected | Not detected | 0.02 |
| Nickel (ppm Ni) | ICP | — | Not detected | <0.0491 | Not detected | 0.1 |
| Phosphorus (ppm P) | ICP | 371 | 296 | 295 | 299 | |
| Potassium (ppm K) | ICP | 174 | 156 | 157 | 157 | |
| Silicon (ppm Si) | ICP | — | <4.99 | <4.91 | <4.90 | 0.2 |
| Sodium (ppm Na) | ICP | 3,617 | 3,800 | 3,800 | 3,900 | |
| Sulfur (ppm S) | ICP | — | <4.99 | <4.91 | <4.90 | 0.2 |
| Tin (ppm Sn) | ICP | | Not detected | Not detected | Not detected | 0.3 |
| Zinc (ppm Zn) | ICP | — | Not detected | Not detected | Not detected | 0.1 |
| Chloride (ppm Cl⁻) | ISE | 4,948 | 5,030 | 4,870 | 4,980 | |
| Total organic carbon | A&P | n/a | 35.38 | 31.67 | 31.09 | |
| (ppm) | method No. | | | | | |
| | 9900901 | | | | | |

LOD, limits of detection.
ICP, inductively coupled plasma-optical emission spectrometry,
ISE, ion selective electrode.

Inorganic compounds were measured by Inductively Coupled Plasma Optical Emission Spectrometry using a Thermo Jarrell Ash Iris or equivalent with a cross-flow nebulizer and an autosampler. Plasma conditions were set to the following parameters: Auxiliary Gas, Low; RF Power, 1150; Nebulizer Pressure (PSI), 27.5; Nebulizer Pump-Flush Pump Rate (rpm), 100; Analysis Pump Rate (rpm), 100; Relaxation Time (see), 0.0. Wavelengths employed for analysis, in nanometers, were: Ca, 184.006; Mg, 383.826; Na, 589.892; Fe, 259.940; Cu, 324.754; Mn, 257.610; Si, 288.158; Zn, 213.856; Ba, 483.409; K, 7969.896; Cd, 226.502; Co, 228.616; Cr, 283.563; Nit 231.604; Pb, 220.353; Sn, 189.989. Aliquots of LMP-102™ were loaded onto the machine according to the manufacturer's instructions and the results recorded. See Table 8.

### Example 6

### Verification of Bacteriophage Composition of Mixtures of Bacteriophage

Genomic DNA was isolated from 750 μl of LMP-102™ or from monophage stocks by the phenol-chloroform method (see Current Protocols in Molecular Biology). The optical density of the DNA solutions was measured at 260 nm, and the DNA concentrations were calculated from the optical density measurements at 260 nm using the conversion factor for double-stranded DNA that OD260=1.0=50 μg/ml DNA. The DNA concentrations were adjusted to 10 μg/ml using water. To amplify each phage, primer pairs corresponding to the sequences shown in Table 3 were synthesized by the Biopolymer lab at the University of Maryland, Baltimore, Md. and were provided with concentration data by the vendor. Fifty picomoles of each primer pair per reaction were used.

### Example 7

### Exclusion of Phage Bearing Undesirable Genes and Sequences

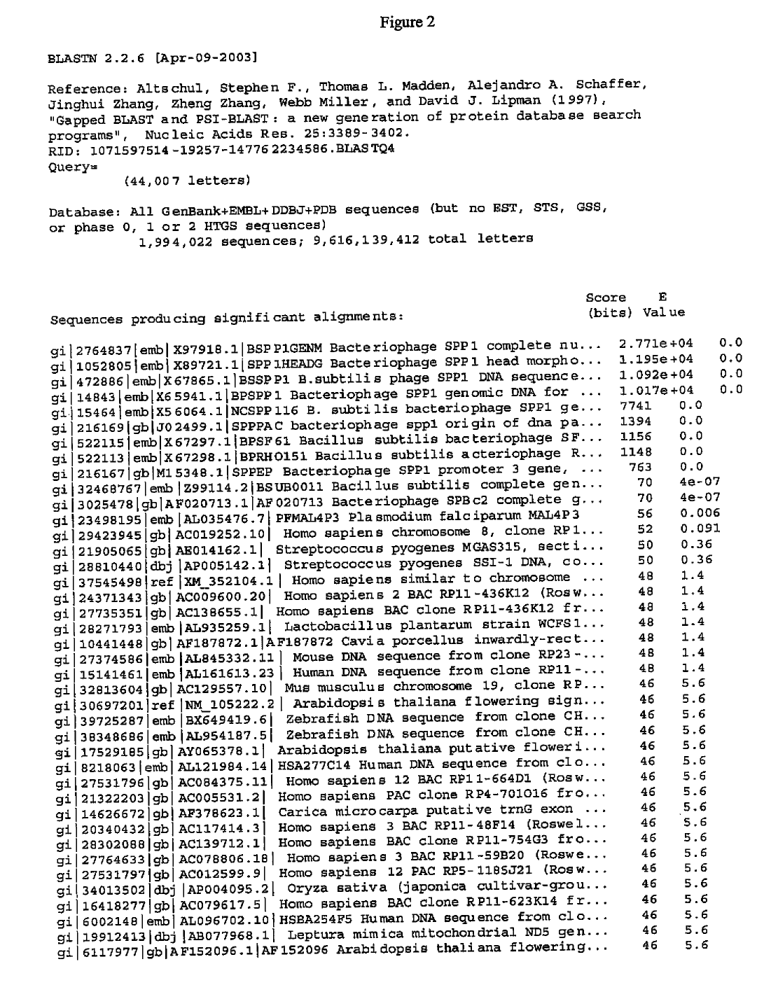

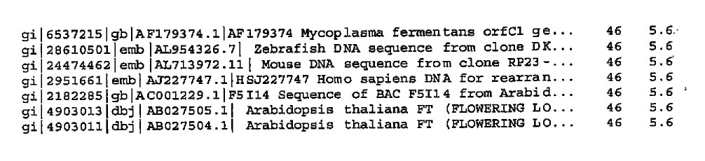

In the following example, the publicly available sequence of Bacteriophage SPP1, GenBank number NC 004166, was searched against the GenBank database using the BlastN program, as referenced in FIG. 2. The data in FIG. 2 show that only 11 sequences yielded e values of ≦10⁻⁵. No bacterial toxin genes are present, and there are no sequences present that are derived from bacterial 16s ribosomal RNA genes.

### Example 8

### Contamination with Bacterial Endotoxin or Other Toxins

Aliquots of LMP-102™, a mixture of six bacteriophages specific for strains of Listeria monocytogenes, were assayed for listeriolysin 0 by the method of Geoffroy (see Geoffroy et al. (1987) Purification, characterization, and toxicity of the sulfhydryl-activated hemolysin listeriolysin 0 from Listeria monocytogenes. Infect Immun. 55: 1641-6.), and for endotoxin by the well-established limulus amoebocyte assay using the QLC-1000 Bio-Whittaker LAL kit according to the manufacturer's directions (Cambrex Corporation, East Rutherford, N.J.). Results are presented in Table 9.

TABLE 9

| | LMP-102 ™ | LMP-102 ™ | LMP-102 ™ | | |
|---|---|---|---|---|---|
| | Lot | Lot | Lot | | Limit |
| Assay | 0103J020142 | 0103J020215 | 0103J020361 | Specification | of detection |
| Listeriolysin O | Not detected | Not detected | Not detected | Not detected | 5 HU/ml |
| Endotoxin | 66 EU/ml | 79 EU/ml | 83 EU/ml | <350 EU/ml | 0.1 EU/ml |

### Example 9

### Measurement of Total Protein, Total Kjeldahl Nitrogen, pH, Specific Gravity, Total Dissolved Solids, Percent Ash, and Total Non-Volatile Solids

Aliquots three lots of LMP-102™, a mixture of six bacteriophages specific for strains of Listeria monocytogenes, were assayed for pH using a calibrated pH meter as is common in the art. Specific gravity was determined by precisely measuring a volume of L-102™, weighing it, and calculating from the volume weighed the weight of a 1 ml sample. Total protein was determined by the method of Lowry (see Lowry O H et al., Protein measurement with the folin phenol reagent. J Biol Chem 193: 265-275 1951) using the Protein Assay Kit #P5656 (Sigma Diagnostics, St. Louis, Mo.) according to the manufacturer's directions. Total Kjeldahl nitrogen was measured according to the methods of O'Dell, McDaniel et al. and Gales & Booth (see McDaniel et al. Automatic determination of Kjeldahl nitrogen in estuarine water. Technicon Symposia 1:362-367, 1967; Gales & Booth Evaluation of the Technicon block digestor system for the measurement of total Kjeldahl nitrogen and total phosphorus. EPA-600/4-78/015, Environmental Monitoring and Support Laboratory, Cincinnati, Ohio; O'Dell Determination of total Kjeldahl nitrogen by semi-automated colorimetry. EPA Method 351.2, Revision 2.0, 1993 Environmental Monitoring Systems Laboratory, Cincinnati, Ohio). Total dissolved solids were determined by drying filtered aliquots of LIP-102™ at 105° C. and 180° C. in a crucible then dividing the weight of the dried residue by the weight of the filtrate to determine the percent dried solids. The percent ash was determined by heating aliquots of LMP-102™ to 600° for 2 to 18 hours until a stable weight is obtained. The weight of the residue divided by the weight of the original aliquot of LMP-102™ yielded the percent ash. Table 10 illustrates the data obtained for three lots. Total non-volatile solids are calculated as indicated in the table.

TABLE 10

| | | | LMP-102 ™ | LMP-102 ™ | LMP-102 ™ |
|---|---|---|---|---|---|
| | | Theoretical | Lot | Lot | Lot |
| Analysis | Method | (1X PBS) | 0103J020142 | 0103J020215 | 0103J020361 |
| pH | pH meter | 7.3-7.5 | 7.41 | 7.37 | 7.42 |
| Specific gravity | DMA46 | 1.007 | 1.005 | 1.005 | 1.005 |
| % Total dissolved solids | 105° C., 24 h | 1.00% | 1.02% | 1.01% | 1.02% |
| % Total dissolved solids | 180° C., 24 h | — | 0.96% | 0.98% | 0.88% |
| % Ash (inorganic solids) | 600° C., 3-12 h | 1.00% | 0.93% | 0.95% | 0.94% |
| % Total non-volatiles, | (TDS₁₀₅-Ash) | n/a | 0.09% | 0.06% | 0.08% |
| 105 | | | | | |
| % Total non-volatiles, | (TDS₁₈₀-Ash) | — | 0.03% | 0.03% | −0.06% |
| 180 | | | | | |
| Total protein (ppm) | Modified Lowry | n/a | 18.1* | 17.6* | 18.0* |
| | assay | | | | |
| Total nitrogen (ppm) | Hach, Test ‘N | n/a | 5.1 | 5.3 | 4.8 |
| | Tube ™ | | | | |
| Total Kjeldahl nitrogen | EPA 351.2 | n/a | 5.56 | 6.26 | 5.41 |
| (ppm) | | | | | |

### Example 10

### Determination of Bacteriophage Morphology and Homogeneity by Electron Microscopy

Electron microscopy was performed on each of the six monophages comprising LMP-102™ by the method of Carlson (see Carlson, L (1994) Visualization of T4 phage by electron microscopy. In Molecular Biology of Phage T4. Karam (eds.). Washington, D.C.: ASM Press, pp. 482-483.). The electron micrographs were used to assess homogeneity qualitatively and to determine the dimensions of each bacteriophage, shown in Table 2.

We claim:

1. A method for assuring identity of a regulated bacteriophage preparation comprising
a) obtaining at least two phage plaques from said preparation; and
b) comparing nuclease and proteases digests of said plaques with each other and with that of a reference strain;
wherein disparity in relative molecular weight indicates lack of identity.

2. The method of claim 1 further comprising determining the presence of bacteria in said preparation.

3. The method of claim 1 further comprising determining the presence of toxin in said preparation.

4. The method of claim 1, further comprising testing at least one of total protein, total nitrogen, pH, specific gravity, total dissolved solids, percent ash and total non-volatile solids of said preparation.

5. A method of preparing a lytic bacteriophage preparation for therapeutic use comprising
a) growing a population of bacteriophage in a culture of host bacteria;
b) removing host bacteria from the population of bacteriophage;
c) producing a monoclonal population of said bacteriophage;
d) testing the population of bacteriophage for bacterial contamination;
e) testing the population of bacteriophage for endotoxin contamination; and
f) testing the population of bacteriophage for contamination by lysogenic phage.

6. A phage selected from the group consisting of List 1, List 2, List 3, List 4, List 36 and List 38.
